(19) United States
(12) Patent Application Publication
(10) Pub. No.: US 2013/0318866 A1
(43) Pub. Date: Dec. 5, 2013

(54) Apparatus And Method For Biological Growth Enhancement

(71) Applicant: Robert Dale Gunderman, JR., Honeoye Falls, NY (US)

(72) Inventor: Robert Dale Gunderman, JR., Honeoye Falls, NY (US)

(21) Appl. No.: 13/906,082

(22) Filed: May 30, 2013

Related U.S. Application Data
(60) Provisional application No. 61/654,901, filed on Jun. 3, 2012.

(51) Int. Cl. C12N 13/00; A01C 1/00

(52) U.S. Cl. CPC C12N 13/00; A01C 1/00
USPC 47/1.3

(57) ABSTRACT

An apparatus and method for biological growth enhancement is disclosed. Organisms that will benefit from the apparatus and method of the present invention include seeds, fungus, bacteria, and the like. In one example, seeds are hydro-primed, exposed to a high voltage electric field, and prepared for germination. The resulting sprouts are larger than those that have not been treated by the apparatus for biological growth enhancement. In addition, the root systems of sprouts treated by the apparatus for biological growth enhancement were more advanced than those that were not treated. Benefits include increased production rate of edible sprouts, seedlings that are able to withstand adverse conditions such as drought at an earlier age, and a reduction in the resources required to grow sprouts and plants.

### CROSS REFERENCE TO RELATED PATENT APPLICATIONS

This application claims priority to U.S. Patent Application Ser. No. 61/654,901 filed Jun. 3, 2012 entitled “System, Apparatus And Method For Electrostatically Enhanced Seed Priming And Treatment”. The disclosure of this U.S. Patent Application Ser. No. 61/654,901 is incorporated herein by reference in its entirety.

### BACKGROUND OF THE INVENTION

1. Field of the Invention

This invention relates generally to agriculture, and more specifically to an apparatus and method for the growth enhancement of biological organisms such as seeds, seedlings, sprouts, plants, fungi, and bacteria.

2. Description of Related Art

The germination and growth of seeds is of vital importance to the ongoing viability of human civilization. Over the years, there have been various attempts to improve factors such as germination percentage, speed of germination, hardiness of seedlings, survivability of seedlings in unfavorable conditions, and the like. When a seed is planted in less than ideal growing conditions, there are many ways in which an emerging seedling can be damaged or destroyed. Drought poor soil and nutrients, birds, insects, fungus, and other such maladies all play a factor in poor crop yield. Lack of a proper and adequate crop yield in many parts of the world can mean starvation, poor nutrition, and related illnesses. Advancing a young seedling into a strong and thriving plant as quickly as possible greatly improves crop yield and improves the wellbeing of the grower and their community. Various techniques such as fertilizers, fungicides, pesticides, row covers, increased watering, and the like all help in seedling growth and development; getting to a strong, thriving and producing plant quicker. One such simple technique is that of hydro-priming. Hydro-pruning is simply soaking a seed in water for a period of time before planting. The seed will swell with water and germinate quicker. Any technique that provides a stronger and larger seedling in a shorter amount of time has great value in agriculture. Besides hydro-priming and chemical treatments such as fertilizers, pesticides, and fungicides, there have been few advances in techniques to improve germination time and seedling vigor that are not chemical based. The ability to enhance the growth rate of seedlings has important implications for agriculture. A more vigorous and advanced seedling is better able to withstand drought conditions and other environmental conditions that are detrimental to seedling and plant growth. Growth enhancement of seedlings also means a more advanced root system that is able to extract nutrients and water more efficiently.

In addition to field crops, the growth of sprouts such as alfalfa sprouts, mung bean sprouts, pea sprouts, and the like, is a crop unto itself. Sprouts are used in salads, stir fry, and other prepared dishes. To increase the production of sprouts by a grower, more building space is needed, as the growth time from seed to marketable product has heretofore been fixed. Increased building space also must include increased electric and heat usage, increased water use, real estate taxes, increased maintenance equipment costs, and the like.

Other organisms, such as fungi and bacteria, would also benefit from an apparatus and method to enhance growth rates. For example, fungi are grown for many purposes. Edible mushrooms are grown commercially, with some varieties taking many months to mature. Fungi are also grown to produce chemicals used for pharmaceuticals and industrial applications. A well known example of the use of a fungus for pharmaceutical production is that of the penicillium fungi, which is used to produce penicillin.

### BRIEF SUMMARY OF THE INVENTION

In accordance with the present invention, there is provided an apparatus for biological growth enhancement comprising an enclosure having a top, a bottom, three sides and an access door; a first electrode having a generally planar form and disposed within said enclosure; a second electrode spaced apart from said first electrode; electrode adjustment points to vary the spacing between the first electrode and the second electrode; an electronics module comprising a variable output high voltage power supply having a two conductor output; the two conductor output of the high voltage supply being electrically connected to the first electrode and the second electrode respectively; and at least one removable tray for retaining organisms to be treated; whereas the removable tray is sized to fit within the enclosure and between the first electrode and the second electrode.

The foregoing paragraph, has been provided by way of introduction, and is not intended to limit the scope of the invention as described in this specification, claims and the attached drawings.

### BRIEF DESCRIPTION OF THE DRAWINGS

The invention will be described by reference to the following drawings, in which like numerals refer to like elements, and in which;

FIG. 1 is a plan view of an exemplary embodiment of a seed priming and treatment system of the present invention;

FIG. 2 is a process flowchart depicting a method of the present invention;

FIG. 3 is a plan view of an exemplary seed priming and treatment system of the

present invention performing a hydro-priming step;

FIG. 4 is a plan view of the seed priming and treatment system depicted in FIG. 3 evacuated of water and ready for electrostatic treatment of seeds;

FIG. 5 is a perspective view of a growth enhancement apparatus of the present invention;

FIG. 6 is a top plan view of the growth enhancement apparatus depicted in FIG. 5;

FIG. 7 is a front plan view of the growth enhancement apparatus depicted in FIG. 5;

FIG. 8 is a bottom plan view of the growth enhancement apparatus depicted in FIG. 5;

FIG. 9 is a sectional view of the growth enhancement apparatus depicted in FIG. 5 taken along line A-A of FIG. 7;

FIG. 10 is a side plan view of the growth enhancement apparatus depicted in FIG. 5;

FIG. 11 is an alternate side plan view of the growth enhancement apparatus depicted in FIG. 5;

FIG. 12 is a front plan view of the growth, enhancement apparatus depicted in FIG. 5 with the door removed;

FIG. 13 is a perspective view of the growth enhancement apparatus depicted in FIG. 5 with the door and top removed;

FIG. 14 is an electrical diagram of the growth enhancement apparatus of FIG. 5; and

FIG. 15 is a method of determining optimal treatment parameters for the growth enhancement apparatus of FIG. 5.

The present invention will be described in connection with a preferred embodiment, however, it will be understood that there is no intent to limit the invention to the embodiment described. On the contrary, the intent is to cover all alternatives, modifications, and equivalents as may be included within the spirit and scope of the invention as defined by this specification, claims and the attached drawings.

### DESCRIPTION OF THE PREFERRED EMBODIMENTS

Disclosed herein is an apparatus and method for biological growth enhancement. An example of biological growth enhancement, as used herein, is that of seed priming and treatment. This specification discloses a growth enhancement system as well as an exemplary seed priming and treatment system, and related methods thereof, it should be noted that the growth enhancement system of the present invention, while well suited for seed priming and treatment may also be used to enhance the growth of other organisms such as fungi, bacteria, and the like. The treatment parameters of the growth enhancement system of the present invention may vary based on the target organism, and the results desired. For example, the voltage and exposure time needed for Mung bean seeds to produce robust sprouts may be different from the voltage and exposure time needed for wheat seeds to produce robust root systems. And the voltage and exposure time for Reishi mushrooms may again be different than that of various seeds.

There are various techniques for the design and construction of a growth enhancement system of the present invention. The shape, size, materials and components selected for the growth enhancement system may vary based on the intended application. Such adaptations and modifications will become evident to one skilled in the art after reading this specification and viewing the attached drawings.

For a general understanding of the present invention, reference is made to the drawings. In the drawings, like reference numerals have been used throughout to designate identical elements.

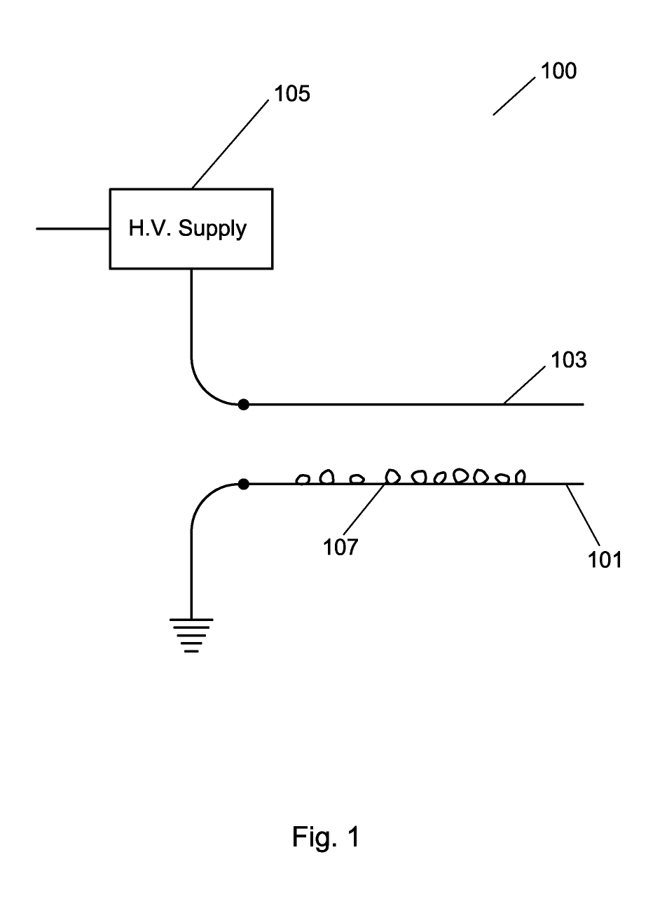

FIG. 1 is a plan view of an exemplary embodiment of a seed priming and treatment system of the present invention 100. Depicted in FIG. 1 is a first electrode 101 and a second electrode 103. The first electrode 101 is electrically connected to ground (for example, but not limited to the ground reference of the high voltage supply 105), and the second electrode 103 is electrically connected to the high voltage supply 105. In some embodiments of the present invention, the second electrode 103 is connected to a negative high voltage supply, and in other embodiments of the present invention the second electrode 103 is connected to a positive high voltage supply. In some embodiments of the present invention, the second electrode 103 is connected to a negative high voltage supply, and in other embodiments of the present invention the second electrode 103 is connected to a positive high voltage supply. The order and naming of the first and second electrodes is arbitrary, where the second electrode 103 may be the ground connection. The high voltage supply 105 may be a simple buck-boost circuit to provide high voltage, or it may be a commercial high voltage power supply such as the high voltage supplies manufactured by Emco High Voltage, Inc. In one embodiment of the present invention, the voltage provided by the high voltage power supply 105 may be in the range of 3,000-7,000 volts D.C., however, other high voltage values may also be suitable. In some embodiments of the present invention, the high voltage power supply 105 may provide pulsed D.C. or A.C., or high voltage with a frequency component, or various high voltage waveforms and frequencies with various associated current components. In some embodiments of the present invention, the high voltage provided to the electrodes is electrostatic, where there is low current but high voltage and there is no arcing or related breakdowns such as corona discharge. In other embodiments of the present invention, arcing or corona discharge may be applied either for the duration of the treatment of the seeds 107, or for a portion of the electrostatic treatment time with the remainder of the electrostatic treatment time being electrostatic. As seen in FIG. 1, the seeds 107 are resting on the first electrode 101. Seeds 107 may also be, in some embodiments of the present invention, other biological material such as fungi or bacteria. The fungi may be in the form of mycelium, or may include fruiting bodies and other features. The fungi may also be in the form of spores, in some embodiments of the present invention, the seeds 107 may be placed on a suitable seed holder that may electrically or mechanically a part of the first electrode 101, or the seed holder may be placed between the first electrode and the second electrode. In some embodiments of the present invention, one or both of the electrodes may be a part of the apparatus itself, such as, for example, a metal or partially metal housing being adapted to serve as an electrode through appropriate electrical fittings and proper insulation from the other electrode. The seed priming and treatment system 100 allows the seeds 107 to be exposed to a high voltage electric field for a specified time interval. The seeds 107, prior to being exposed to the high voltage electric field, may be hydro-primed. Hydro-priming involves soaking the seeds in water for a specified period of time to allow the seed to swell and uptake water. Hydro-priming may be performed within the apparatus of the present invention, or may be performed outside the apparatus of the present invention with the seeds 107 subsequently being placed between the electrodes for electrostatic treatment. Without being bound to any one particular theory, water, being a polar molecule, can be moved by electrostatic forces. It is believed that electrostatic treatment of a seed after, or in conjunction with hydro-priming moves water into the seed in ways that are beneficial to germination and seedling vigor. Thus, a form of enhanced and improved hydro-priming is provided that improves seedling germination rate, reduces germination time, and improves seedling vigor. As can be envisioned after reading this disclosure, the apparatus depicted in FIG. 1 teaches the basic requirements for the present invention, namely a high voltage electric field, provisions to place seeds in the generated high voltage electric field, and in some embodiments of the present invention, provisions to hydro-prime the seeds. In some embodiments of the present invention, hydro-priming may be omitted and the dry seed treated with high voltage prior to growth. A timer to control the high voltage exposure time may also be included in some embodiments of the present invention.

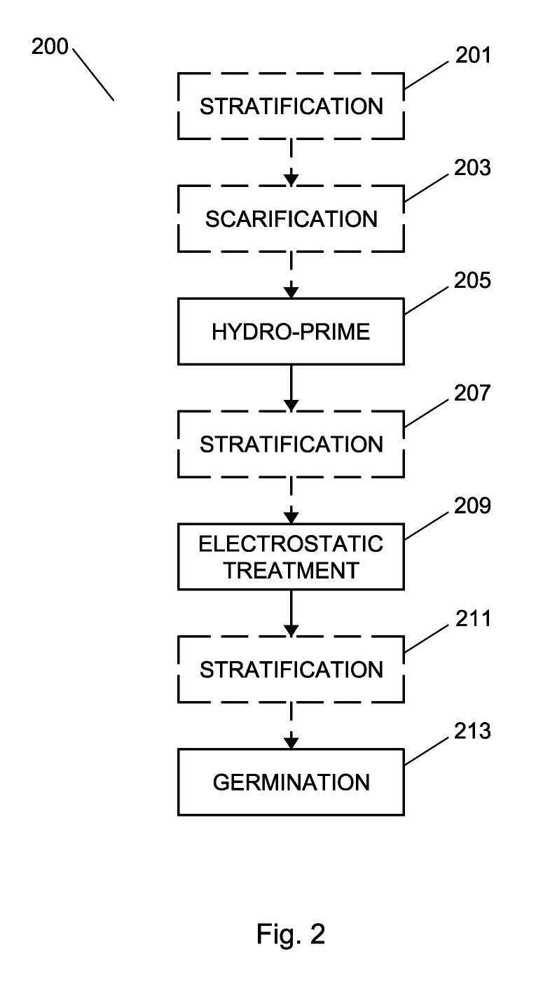

FIG. 2 is a process flowchart depicting a method of the present invention 200. The basic premise is to expose seeds that have been hydro-primed to a high voltage electric field. FIG. 2 depicts several optional steps of stratification and scarification that may be required or suitable for certain seed types. Stratification involves cooling the seed for a period of time, and scarification Involves mechanically abrading, cutting or nicking the hard outer layering of some seeds. In optional step 201, a seed undergoes stratification, and then in optional step 203 the seed undergoes scarification. The seed undergoes hydro-priming (hydro-prime) in step 205. Hydro-priming involves soaking the seed for a specified period of time that may be seconds, minutes, hours, or even days. The seed may then undergo an optional stratification step 207, and then undergo electrostatic treatment 209 where the seed is exposed to a high voltage electric field for a specified time interval. Electrostatic treatment 209 has been previously described by way of accompanying FIG. 1, and may, in some embodiments of the present invention, contain an arcing or corona discharge component for a portion of the electrostatic treatment. The seed may undergo an optional stratification step 211 and then undergo a germination step 213 where the seed is placed in a suitable growing medium such as soil and allowed to germinate. It should be noted that in some embodiments of the present invention, the seeds may be hydro-primed, electrostatically treated, and then stored or further processed to delay start of germination to accommodate factors such as shipping, inclement weather, or the like. Further processing to delay germination may include steps such as, for example, lowering the moisture content, cooling the seeds, or the like.

In April of 2012, several experiments were conducted at a private research lab in the Lennox Tech Center, Rochester, N.Y. to determine the impact of the methods of the present invention on germinating seeds. The test system comprised a system similar to that depicted in FIG. 1. Green Bean seeds (Earliserve Bush from Livingston Seed Company) were hydro-primed for 24 hours before undergoing electrostatic treatment, 16 hydro-primed green bean seeds were exposed to 7,000 volts for 7 minutes with an electrode spacing of 3.5 cm., and then placed on a wet cloth in a Petri dish, where the water in the Petri dish contained 10-15-10 fertilizer. As a control, 16 hydro-primed green bean seeds from the same lot (no electrostatic treatment) were also simultaneously placed on a wet cloth in a Petri dish where the water in the Petri dish contained 10-15-10 fertilizer. Growing conditions for both sets was the same. In two days, in the group of electrostatically treated seeds, all seeds had sprouted and there were 6 of the total of 16 seeds that possessed a reticle greater than 0.3 inches long. By contrast, the control set (no electrostatic field exposure) had only 2 of the 16 total seeds with reticles greater than 0.3 inches long. After five days, all of the green bean seeds were sprouted. Generally, by visual Inspection, the green bean seeds that were electrostatically treated after hydro-priming had slightly thinner reticles than the green bean seeds that were not electrostatically treated.

In another experiment using Mung Bean seeds, 5 grams of mung bean seeds were hydro-primed for 29.5 hours, and 5 grams of mung bean seeds (control) were also hydro-primed for 29.5 hours. The mung bean seeds had sprouted by 24 hours in both instances. At 29.5 hours, the 5 grams of hydro-primed mung bean seeds that, had sprouted were exposed to 7,000 volts for 7 minutes with an electrode spacing of 3.5 cm. The control of 5 grams of hydro-primed mung bean seeds was not exposed to any high voltage electric field. Both the electrostatically treated mung bean seeds and the control group of mung bean seeds were maintained in separate Petri dishes on moist tissue paper with identical growing conditions next to each other. In 72 hours, it was observed that the electostatically treated mung bean sprouts were more advanced, with longer and generally larger sprouts, lire sprouts were allowed to grow for one week, and each day it was observed that the electrostatically treated sprouts were larger and generally more vigorous. Photos were taken to document this observed difference.

In a similar experiment using alfalfa seeds, 0.5 grams of alfalfa seeds were hydro-primed for 29.5 hours, and 0.5 grams of alfalfa seeds (control) were also hydro-primed for 29.5 hours. The alfalfa seeds sprouted by 24 hours in both instances. At 29.5 hours, the 5 grams of hydro-primed alfalfa seeds that had sprouted were exposed to 7,000 volts for 7 minutes with an electrode spacing of 3.5 cm. The control of 5 grams of hydro-printed alfalfa seeds was not exposed to any high voltage electric field. Both the electrostatically treated alfalfa seeds and the control group of alfalfa seeds were maintained in separate Petri dishes on moist tissue paper with identical growing conditions next to each other. In 72 hours, it was observed, that the electostatically treated alfalfa seed sprouts were more advanced, with longer and generally larger sprouts. The sprouts were allowed to grow for one week, and each day it was observed that the electrostatically treated sprouts were larger and generally more vigorous. Photos were taken to document this observed difference.

Further experimentation continued, and in January of 2013 wheat seeds were treated for five and ten minutes at 3 kilovolts, 6 kilovolts, and 11 kilovolts using the same experimental setup as before. The electrode spacing was 3.5 cm. and a control of 0 kilovolts was also used. Each sample set consisted of 200 wheat seeds in a Petri dish with adequate water. The seeds were soaked overnight before treatment. Six clays later, there was a noticeable height difference in the wheat grass sprouts, with 3 kilovolts for 5 minutes resulting in approximately 4.5 cm. sprouts, and 6 kilovolts for 10 minutes resulting in approximately 4.5 cm. sprouts. While the control (no voltage) produced sprouts of approximately 3.0 cm. and exposure to 11 kilovolts for five and ten minutes resulted in approximately 2.5 cm. sprouts. The sprouts exposed to 3 kilovolts for 5 minutes and 6 kilovolts for 10 minutes also had more extensive root systems.

The ability to hydro-prime and electrostatically treat seeds in a single apparatus that may, in some embodiments, be a continuous process, may be desirable for some applications such as, for example, commercial growing operations, operations that lack skilled operators, operations that require enhanced safety or simplicity, and the like. Many continuous process or self-contained devices may be envisioned after reviewing this disclosure and the accompanying drawings, and are to be considered within the spirit, and broad scope of the present invention.

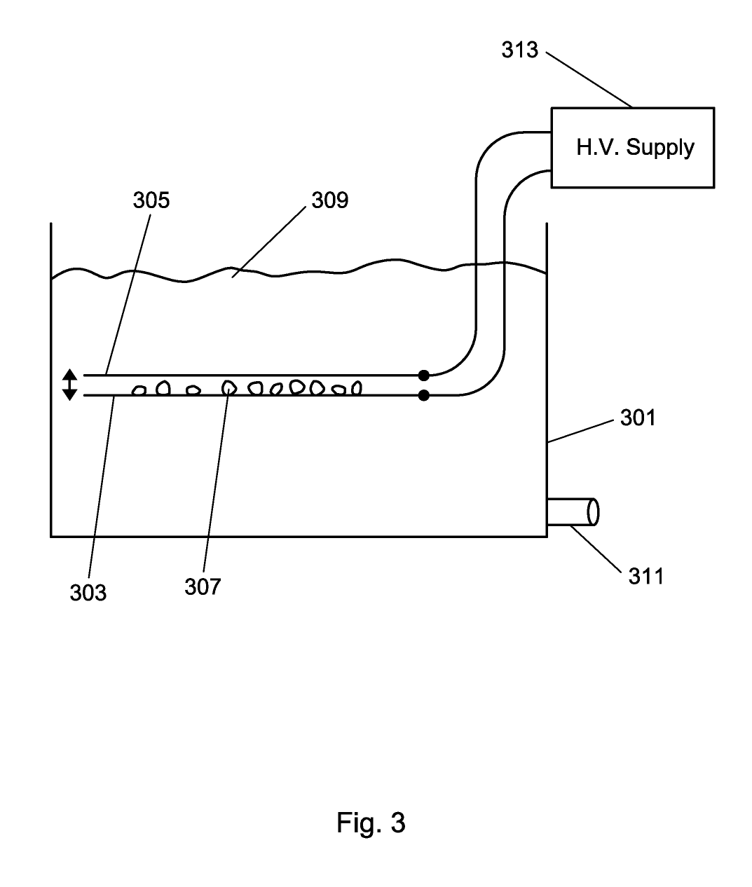

For example, FIG. 3 is a plan view of an exemplary seed priming and treatment system of the present invention performing a hydro-priming step. A vessel 301 or other suitable container is depicted that Is capable of retaining water. The vessel 301 may, in some embodiments, have a water connection 311 that may provide water fill or water drain capabilities. The water connection 311 may comprise more than one connection, or be placed in various locations on or within the vessel 301. in FIG. 3, the vessel 301 is filled with water 309, and the first electrode 303 and the second electrode 305 are submerged in the water 309. The electrodes act to retain the seeds 307 while being hydro-primed, and may, in some embodiments of the present invention, be in close proximity to each other to ensure that the seeds stay in place in the water, and do not float or otherwise move from their intended location. The high voltage supply 313 is appropriately connected to the electrodes, as previously described herein, and may have various electrical performance characteristics, also as previously described herein. The first electrode 303 is electrically connected to ground (for example, but not limited to, the ground reference of the high voltage supply). In some embodiments of the present invention, the second, electrode 305 is connected, to a negative high, voltage supply, and in other embodiments of the present invention the second electrode 305 is connected to a positive high voltage supply. The order and naming of the first and second electrodes is arbitrary, where the second electrode 305 may be the ground connection in some embodiments of the present invention. The high voltage supply may be a simple buck-boost circuit to provide high voltage, or it may be a commercial high voltage power supply such, as the high voltage supplies manufactured by Emco High Voltage, Inc. In one embodiment of the present invention, the voltage provided by the high voltage power supply may be in the range of 3,000-7,000 volts D.C. In some embodiments of the present invention, the high voltage power supply may provide pulsed D.C., or A.C., or high voltage with a frequency component, or various high voltage waveforms and frequencies with various associated current components. In some embodiments of the present invention, the high voltage provided to the electrodes is electrostatic, where there is low current but high voltage and there is no arcing or related breakdown such as corona discharge. In other embodiments of the present invention, arcing or corona discharge may be applied either for the duration of the electrostatic treatment of the seeds 307, or for a portion of the electrostatic treatment time with the remainder of the electrostatic treatment time being electrostatic. As seen in FIG. 3, the seeds 307 are resting on the first electrode 303. In some embodiments of the present invention, the seeds 307 may be placed on a suitable seed holder that may be electrically or mechanically a part of the first electrode 303, or the seed holder may be placed between the first electrode and the second electrode. In some embodiments of the present invention, one or both of the electrodes may be a part of the apparatus itself, such as, for example, a metal or partially metal housing being adapted to serve as an electrode through appropriate electrical fittings and proper insulation from the other electrode.

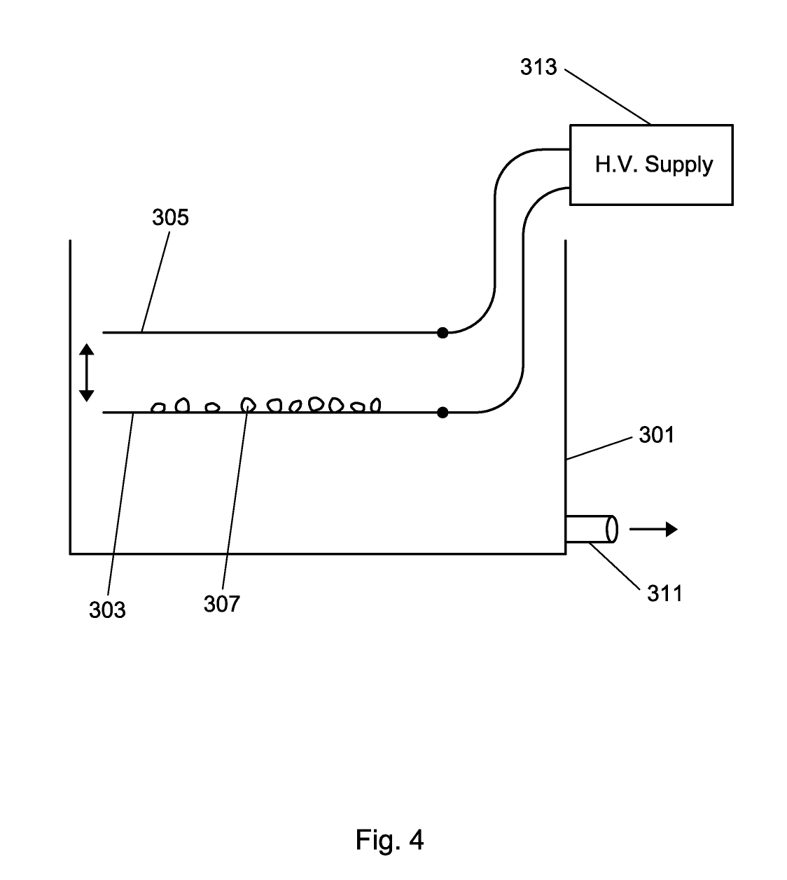

FIG. 4 is a plan view of the Seed Priming and Treatment system depicted in FIG. 3 evacuated of water and ready for electrostatic treatment of seeds. The spacing of the first electrode 303 and the second electrode 305 has increased to ensure that shorting between electrodes and subsequent voltage drop or voltage instability does not occur. The spacing may be increased through any of a variety of means, including mechanical stops, gears, screws, or the like. In addition, in some embodiments of the present invention, the spacing between electrodes may change by way of an appropriate motor or actuator and associated mechanical components. As one can envision, such a continuous process may be further automated through a suitable microprocessor based control system that controls such functions as water flow and evacuation, electrode spacing, electrostatic dwell time, hydro-priming time, temperature, and the like.

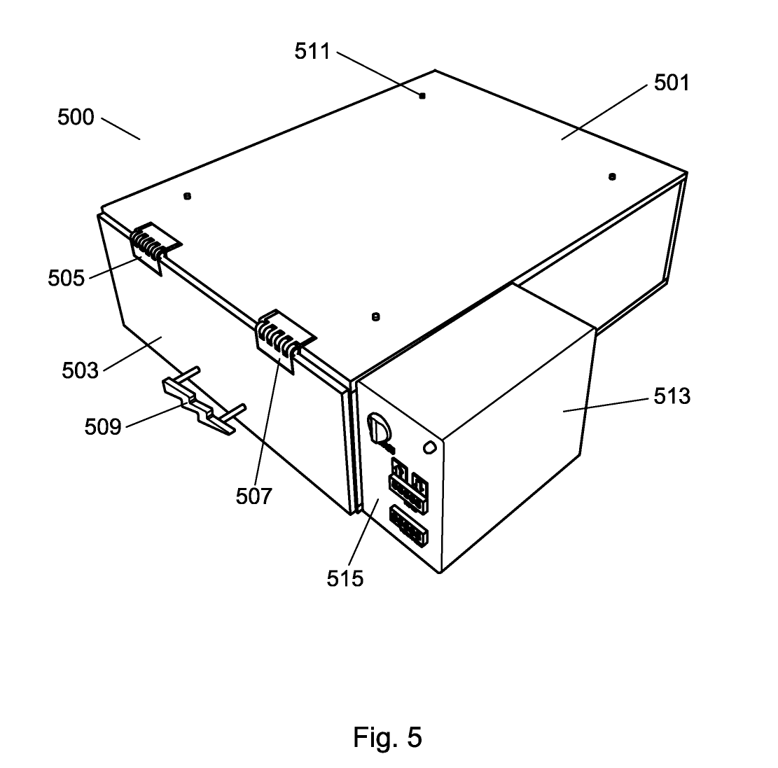

FIG. 5 is a perspective view of a growth enhancement apparatus 500 of the present invention. The apparatus for biological growth enhancement comprises an enclosure 501 having a top, a bottom, three sides and an access door 503. The access door 503 is removably connected to the enclosure 501 with hardware such as a first hinge 505 and a second hinge 507, and may also include an access door handle 509. A safety interlock circuit may, in some embodiments of the present invention, be present where a mechanical switch is employed between the access door 503 and the enclosure sides. The enclosure 501 is preferably non-conductive, and may be made from any of a variety of materials, for example, plastics. Examples of suitable plastics include acrylonitrile butadiene styrene (ABS), polyethylene, polypropylene, polystyrene, polyvinyl chloride, polytetrafluoroethylene, and the like. Bioplastics may also be used in some embodiments of the present invention. In addition, reinforced plastics, and other materials that may be suitably formed may also be used. The enclosure 501 may be made by injection molding, blow molding, machining, extruding and assembling, or the like. Disposed within the enclosure 501 is a first electrode 903 (see FIG. 9) having a generally planar form. A second electrode 905 is spaced apart from said first electrode 903. In some embodiments of the present invention, the second electrode 905 is embedded in or otherwise attached to or made a part of the enclosure 501. The electrodes may be made from any suitable conductive material such as stainless steel, copper, brass, or the like. As depicted in FIG. 9, electrode adjustment points 907, such as slots in at least one side of the enclosure, accommodate the first electrode and allow for adjustment of the spacing between the two electrodes, and thus, the field strength.

In some embodiments of the present invention, retainer protrusions 511 are employed to securely stack multiple growth enhancement apparati. The retainer protrusions 511 may be bumps or similar raised features that engage with a negative of that feature on another growth enhancement apparatus.

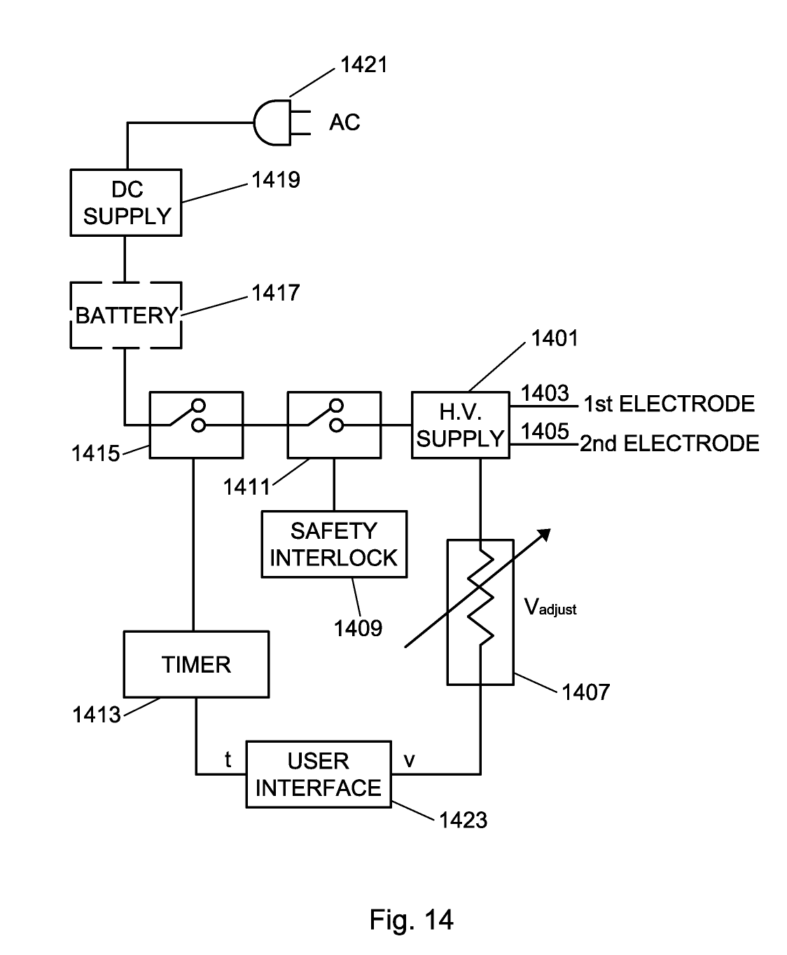

The growth enhancement apparatus further comprises an electronics module 513 having a variable output high voltage power supply having a two conductor output, the two conductor output of the high voltage supply being electrically connected to the first electrode 903 and the second electrode 905 respectively. Details of the electronics module 513 can be seen in FIG. 14. A user interface 515 can be seen in FIG. 5 attached to the electronics module 513. The user interface 515 may include voltage output and time adjustment settings, and may, in some embodiments of the present, invention, include a display and associated functionality.

At least one removable tray 901 for retaining organisms to be treated is sized to fit within the enclosure 501 and between the first electrode 903 and the second electrode 905. The removable tray 901 can be seen in FIG. 9. The removable tray 901 may be made from any of a variety of materials such as plastics, for example. Examples of suitable plastics include acrylonitrile butadiene styrene (ABS), polyethylene, polypropylene, polystyrene, polyvinyl chloride, polytetrafluoroethylene, and the like. In some embodiments of the present invention, the removable tray 901 has drain holes, retention ridges, or other features. The removable tray 901 may accommodate seeds alone, growth medium with seeds or spores, or the like. Once the tray 901 is inserted into the apparatus for biological growth enhancement, the access door 503 is closed, activating the safety interlock circuit and allowing the electrodes to become energized for the duration and voltage specified on the electronics module user interface 515. Should the access door 503 be opened inadvertently during high voltage treatment, the safety interlock circuit will disable the high voltage power supply. In addition, the high voltage power supply should be current limited for an additional level of safety.

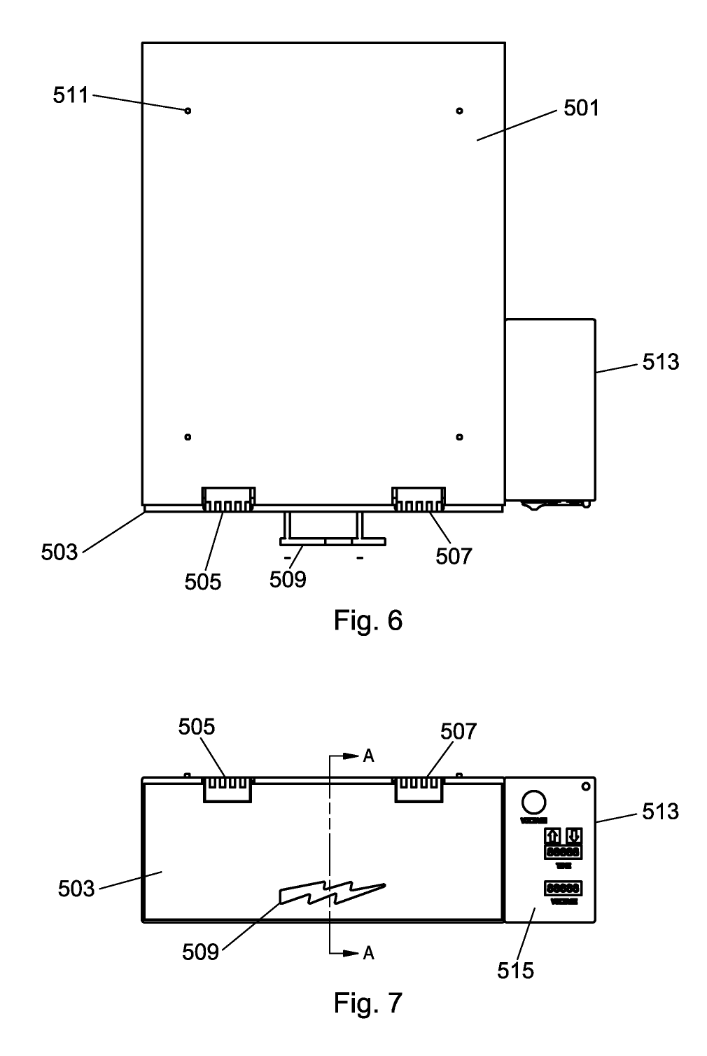

FIG. 6 is a top plan view of the growth enhancement apparatus depicted in FIG. 5 showing clearly the retainer protrusions 511 used to stack multiple biological growth enhancement apparati.

FIG. 7 is a front plan view of the growth enhancement apparatus depicted in FIG. 5. The access door 503 and related hardware can be clearly seen. In some embodiments of the present, invention, the access door 503 is a clear material such as glass or acrylic to allow a user to view the contents of the enclosure 501.

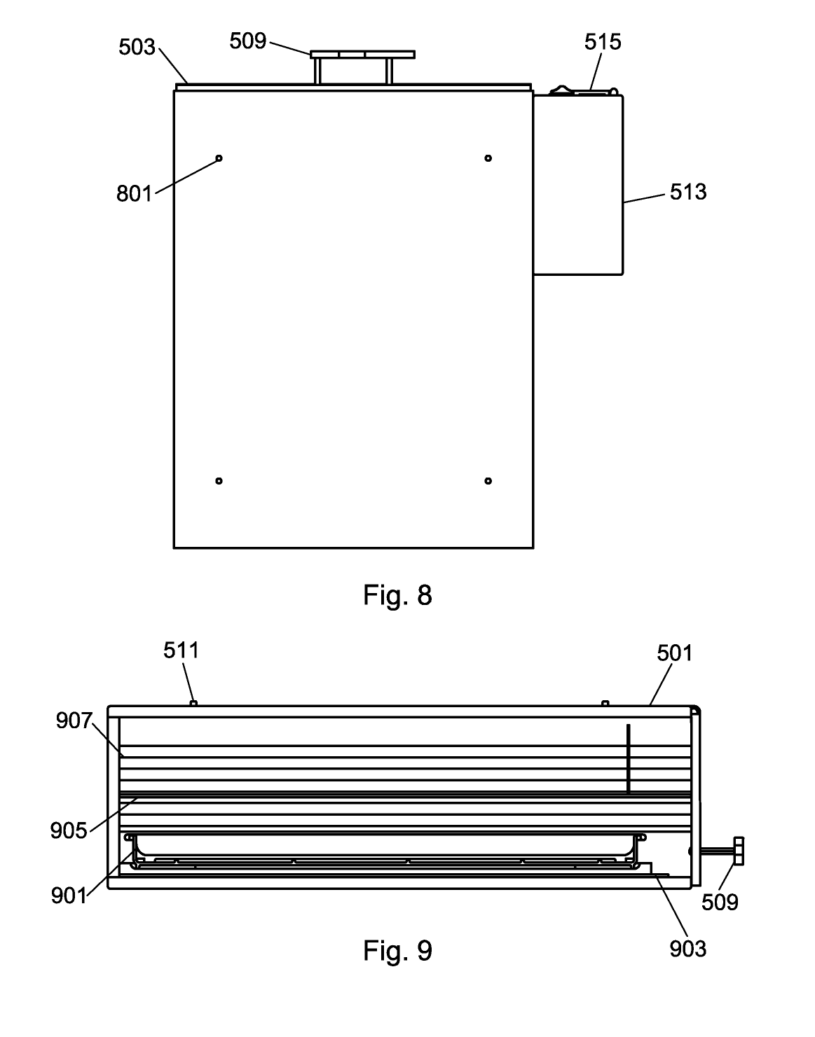

FIG. 8 is a bottom plan view of the growth enhancement apparatus depicted in FIG. 5. Retainer recesses 801 can be seen. The retainer recesses mate with the retainer protrusions 511 on the top of another apparatus to secure a stack of multiple growth enhancement apparati.

FIG. 9 is a sectional view of the growth enhancement apparatus depicted in. FIG. 5 taken along line A-A of FIG. 7. The tray 901 can be seen between the first electrode 903 and the second electrode 905. Electrode adjustment points 907 can be seen along the sides of the enclosure 501, thus allowing the electric field strength to be varied by manipulation of the distance between the first and second electrode.

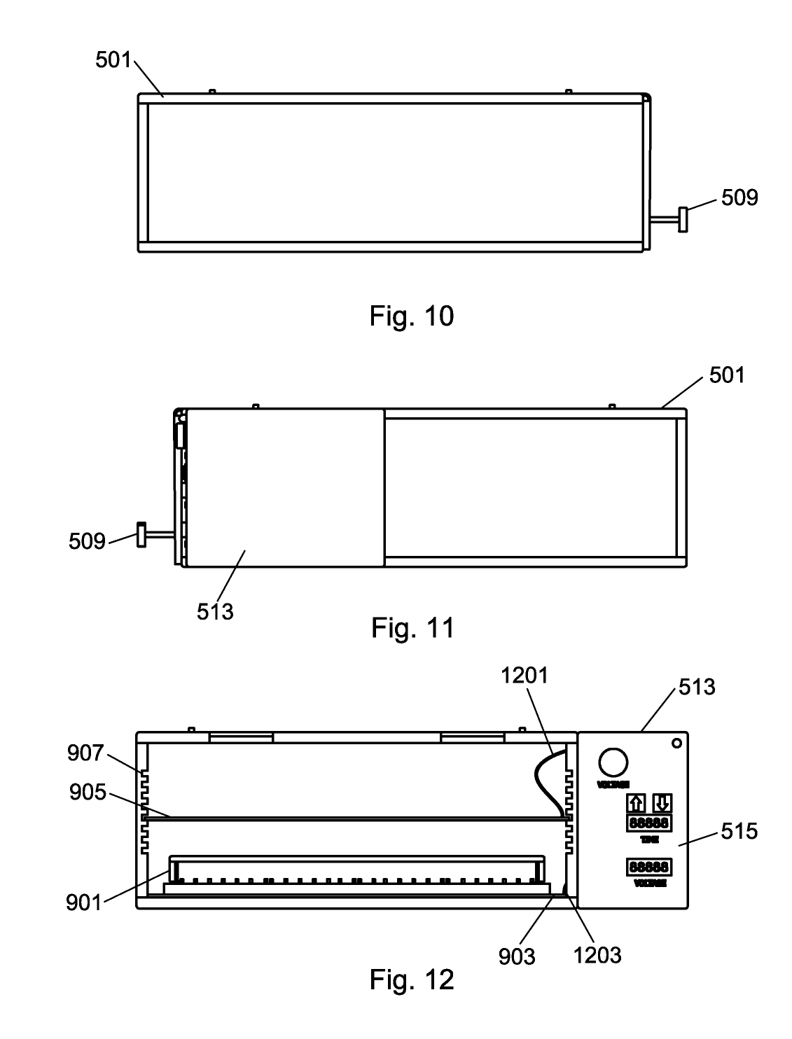

FIG. 10 is a side plan view of the growth enhancement apparatus depicted in FIG. 5 and FIG. 11 is an alternate side plan view of the growth enhancement apparatus depicted in FIG. 5.

FIG. 12 is a front plan view of the growth enhancement apparatus depicted in FIG. 5 with the door removed, showing the tray 901 between the first electrode 903 and the second electrode 905. A second electrode electrical connection 1201 can be seen which connects the second electrode 905 with the high voltage power supply contained within the electronics module 513. A first electrode electrical connection 1203 can also be seen, which connects the first electrode 903 with the high voltage power supply contained within the electronics module 513.

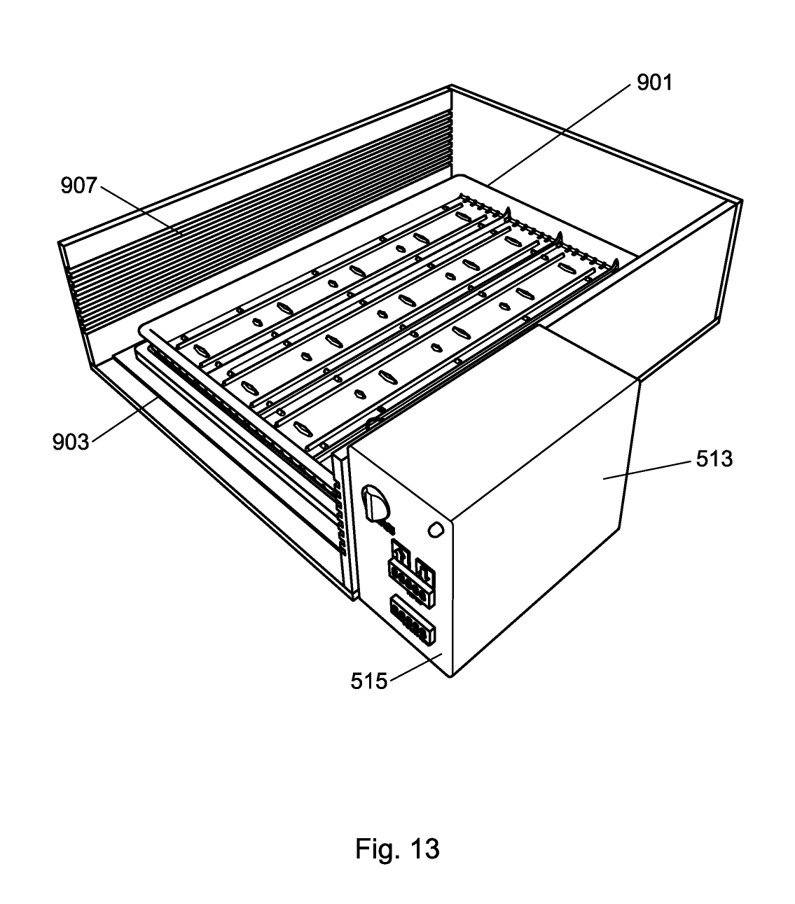

FIG. 13 is a perspective view of the growth enhancement apparatus depicted in FIG. 5 with the door and top removed. The first electrode 903 can be seen below the tray 901. The second electrode 905 is not shown for clarity in FIG. 13.

FIG. 14 is an electrical diagram of the growth enhancement apparatus of FIG. 5. The high voltage supply 1401 may be a simple buck-boost circuit to provide high voltage, or it may be a commercial high voltage power supply such as the high voltage supplies manufactured by Emco High Voltage, Inc. In one embodiment of the present invention, the voltage provided by the high voltage power supply 1401 may be in the range of 1,000-10,000 volts D.C., however, other high voltage values may also be suitable. In some embodiments of the present invention, the high voltage power supply 1401 may provide pulsed D.C., or A.C., or high voltage with a frequency component, or various high voltage waveforms and frequencies with various associated current components. In some embodiments of the present invention, the high voltage provided to the electrodes is electrostatic, where there is low current, but high voltage and there is no arcing or related breakdowns such as corona, discharge. In other embodiments of the present invention, arcing or corona discharge may be applied either for the duration of treatment, or for a portion of the treatment time with the remainder of the treatment time being electrostatic. The high voltage supply 1401 is electrically connected to the first electrode 1403 and the second electrode 1405. The high voltage supply 1401 has a voltage adjustment 1407 that may include a variable resistor. A safety interlock 1409 comprises a switch that is physically connected between the access door 503 and the enclosure 501 (see FIG. 5) that disables the high voltage supply 1401 by way of a switch 1411. The switch 1411, in some embodiments of the present invention, disconnects the low voltage input side of the high voltage supply 1401. In a similar manner, a timer 1413 is connected to a switch 1415 that opens the low voltage input side of the high voltage supply 1401 upon completion of a specified time. The high voltage supply 1401 may, in some embodiments of the present invention, be provided with Direct Current power through a direct current (DC) supply 1419 that may include, in some embodiments of the present invention, a battery 1417. The direct current (DC) supply 1419 may be provided alternating current input power 1421 from a wall receptacle or the like. The battery 1417, should it be included, provides the ability to run the growth enhancement apparatus without connection, to an AC wall outlet. This may be important for operation in a wet environment, for example, a seed sprouting operation. A user interface 1423 Is also provided that may provide voltage and time settings. The voltage output may be adjusted by a simple potentiometer or variable set point resistor that has an analog dial or a digital display and interface. The exposure time may be adjusted by way of timer 1413 using settings in the user interface 1423. Start and stop times and duration may be specified.

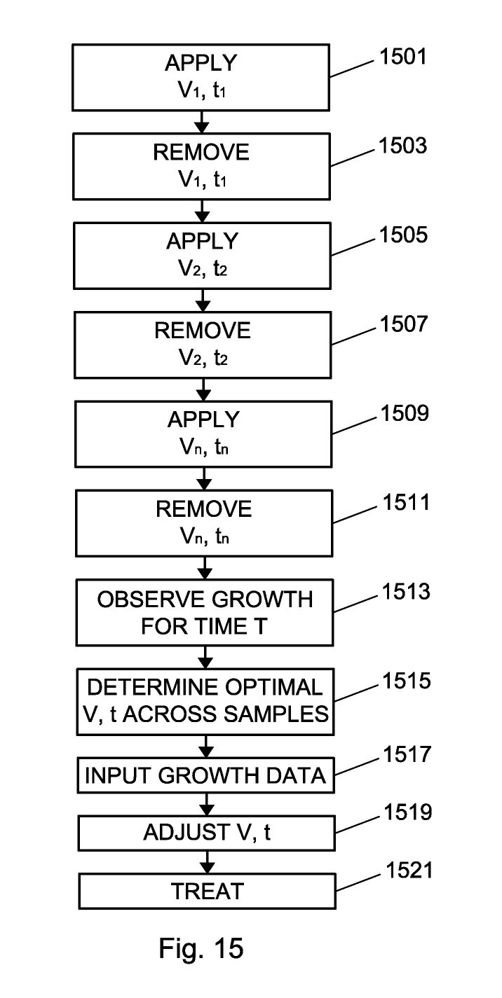

Lastly, it has been found through experimentation that various seed types have different voltage and time settings that are necessary to achieve optimal growth enhancement. A method for determining optimal, treatment parameters for the growth enhancement apparatus is depicted in FIG. 15. A first sample of an organism such, as a seed is provided with a voltage V₁ and an exposure time T₁ in step 1501 and then removed in step 1503. A second sample of the same type of organism is provided with a voltage V₂ and an exposure lime T₂ in step 1505 and then removed in step 1507. Similar steps are repeated in 1509 and 1511 for voltage Vₙ and exposure time Tₙ. Once a number of samples are treated with different voltages and exposure times, growth of the samples is observed for time period T in step 1513. Time Period T may be several days or weeks so that the organism exhibits measurable growth. Once measurable growth is exhibited by the samples, optimal Voltage V and Time t are determined across the samples in step 1515. This growth data is then input to a database or file system in step 1517 and voltage V and Time t are adjusted for the sample being treated in step 1519. Once the optimal voltage V and time T is set in step 1519, the organisms are treated in step 1521.

To use the growth enhancement apparatus 500, organisms such as seeds are placed on the tray 901 (see FIG. 9). The seeds, for example, may be hydro-primed prior to placement on the tray 901, or hydro-priming may take place directly in the tray. The loaded tray 901 is then inserted into the growth enhancement apparatus 500 between, the first and second electrodes. The proper voltage and time is set on the user interface 515, the access door is closed, and the apparatus is activated. Treatment is complete when the timer count is zero, and the access door is opened to remove the loaded tray. The organisms are then placed in their growing environment. In some embodiments of the present invention, subsequent treatments may take place.

It is, therefore, apparent that there has been provided, in accordance with the various objects of the present invention. An Apparatus And Method For Biological Growth Enhancement. While the various objects of this invention have been described in conjunction with preferred embodiments thereof, it is evident that, many alternatives, modifications, and variations will be apparent to those skilled in the art. Accordingly, it is intended to embrace all such alternatives, modifications and variations that fall within the spirit and broad scope of this specification, the attached drawings, and claims.

What is claimed is:

1. An apparatus for biological growth enhancement comprising:
an enclosure having a top, a bottom, three sides and an access door;
a first electrode having a generally planar form and disposed, within said enclosure;
a second electrode spaced apart from said first electrode;
electrode adjustment points to vary the spacing between the first electrode and the second electrode;
an electronics module comprising a variable output high voltage power supply having a two conductor output;
the two conductor output of the high voltage supply being electrically connected to the first electrode and the second electrode respectively; and
at least one removable tray for retaining organisms to be treated; whereas
the removable tray is sized to fit within the enclosure and between the first electrode and the second electrode.

2. The apparatus of claim 1, wherein the electrode adjustment points are slots in at least one side of the enclosure.

3. The apparatus of claim 1, wherein the first electrode is a generally planar stainless steel mesh.

4. The apparatus of claim 1, wherein the second electrode is embedded in the enclosure bottom.

5. The apparatus of claim 1, wherein the electronics module further comprises a high voltage adjustment.

6. The apparatus of claim 1, wherein the electronics module further comprises a timer for setting the high voltage on time.

7. The apparatus of claim 1, wherein the electronics module further comprises a battery for powering the high voltage supply.

8. The apparatus of claim 1, wherein the electronics module further comprises a direct current power supply for powering the high voltage supply.

9. The apparatus of claim 1, wherein the electronics module further comprises a safety interlock circuit.

10. The apparatus of claim 1, wherein the electronics module further comprises a user interface.

11. The apparatus of claim 10, wherein the user interface has set points for voltage and high voltage on time.

12. A method for biological growth enhancement of a seed comprising the steps of:
hydro-priming the seed by soaking the seed in water;
exposing the hydro-primed seed to an electric field;
removing the hydro-primed seed from the electric field; and
providing the hydro-primed seed with conditions favorable for germination and growth.

13. The method of claim 12, wherein the electric field strength is between 100 volts per centimeter and 10,000 volts per centimeter.

14. The method of claim 12, wherein the hydro-primed seed is exposed to an electric field for a period of time between one minute and sixty minutes.

15. The method of claim 12, wherein the seed is hydro-primed in water for between one hour and forty-eight hours.

16. The method of claim 12, further comprising the step of scarification of the seed.

17. The method of claim 12, further comprising the step of stratification of the seed.

18. A method for biological growth enhancement of a fungus comprising the steps of:
placing fungal spores on a growth medium;
providing moisture to the fungal spores;
causing the fungal spores Co grow to fungal mycelium on the growth medium; and
exposing the fungus to an electric field.

19. The method of claim 18, wherein the electric field strength is between 100 volts per centimeter and 10,000 volts per centimeter.

20. The method of claim 18, wherein the mycelium are exposed to an electric field for a period of time between one minute and three months.
